(19) United States
(12) Patent Application Publication
(10) Pub. No.: US 2026/0174025 A1
(43) Pub. Date: Jun. 25, 2026

(54) Systems For Pollinating Plants, And Related Methods

(71) Applicant: Monsanto Technology LLC, Saint Louis, MO (US)

(72) Inventors: Amanuel G. GHEBRETINSAE, Chesterfield, MO (US); Justin Scott KAYSER, Wentzville, MO (US); Louis John MEYER, Creve Coeur, MO (US)

(21) Appl. No.: 19/121,411

(22) PCT Filed: Oct. 17, 2023
(86) PCT No.: PCT/US2023/035350

§ 371 Date: Apr. 15, 2025

Related U.S. Application Data
(60) Provisional application No. 63/417,237, filed on Oct. 18, 2022.

(51) Int. Cl. A01H 5/10; A01G 22/00; A01H 1/02; A01H 5/02; A01H 5/08; A01H 6/54

(52) U.S. Cl. CPC A01H 5/10; A01G 22/00; A01H 1/027; A01H 5/02; A01H 5/08; A01H 6/542

(57) ABSTRACT

Pollination assemblies for use in collecting and transferring pollen between plants are described herein. In one example embodiment, a pollination assembly includes a collection unit configured to dislodge pollen from pollen-bearing plants; a duct unit disposed adjacent the collection unit, wherein the collection unit and the duct unit define a channel extending through the collection unit and the duct unit; and a blower unit configured to direct air into the collection unit and the duct unit to thereby transport the pollen dislodged from the pollen-bearing plants along the channel to pollen-receiving plants adjacent the duct unit.

### CROSS-REFERENCE TO RELATED APPLICATION

This application claims the benefit of, and priority to, U.S. Provisional Patent Application No. 63/417,237, filed Oct. 18, 2022. The entire disclosure of the above application is incorporated herein by reference.

### FIELD

The present disclosure generally relates to systems and methods for pollinating plants.

### BACKGROUND

This section provides background information related to the present disclosure which is not necessarily prior art.

In pollination, pollen grains are typically transferred from male anthers of plants (e.g., of flowers of the plants, etc.) to female stigmas (e.g., of flowers of the plants or other plants, etc.). The plants may be capable of self-pollination, cross-pollination, or both. Self-pollination involves the transfer of pollen from male anthers of plants (e.g., of flowers of the plants, etc.) to female stigmas of the same plants (e.g., of flowers of the same plants, etc.). And, cross-pollination involves the transfer of pollen from male anthers of plants (e.g., of flowers of the plants, etc.) to female stigmas of different plants (e.g., of flowers of different plants, etc.) (e.g., plants from a different family, line, etc.). In this way, the plants are able to create offspring in the form of seeds, which contain genetic information to produce new plants.

### SUMMARY

This section provides a general summary of the disclosure, and is not a comprehensive disclosure of its full scope or all of its features.

Example embodiments of the present disclosure generally relate to pollination assemblies for use in collecting and transferring pollen between plants. In one example embodiment, a pollination assembly generally includes a collection unit configured to dislodge pollen from pollen-bearing plants; a duct unit disposed adjacent the collection unit, wherein the collection unit and the duct unit define a channel extending through the collection unit and the duct unit; and a blower unit configured to direct air into the collection unit and the duct unit to thereby transport the pollen dislodged from the pollen-bearing plants along the channel to pollen-receiving plants adjacent the duct unit.

Example embodiments of the present disclosure also generally relate to pollination systems include the above pollination assembly. In one example embodiment, a pollination system includes a tractor having a carriage; and the pollination assembly coupled to the carriage of the tractor. In another example embodiment, a pollination system includes a rail system having at least one rail and a carriage configured for movement along the at least one rail; and the pollination assembly coupled to the carriage and moveable, via the carriage, along the at least one rail.

Example embodiments of the present disclosure also generally relate to methods for collecting and transferring pollen between plants. In one example embodiment, such a method generally includes receiving at least one pollen-bearing plant into a housing of a collection unit of a pollination assembly; dislodging, by at least one agitator, pollen from the at least one pollen-bearing plant within the housing; directing, by a first air stream, the dislodged pollen from the pollen-bearing plant in an upward direction within the housing; and directing, by a second air stream, the dislodged pollen from the housing into a duct unit adjacent the housing of the collection unit for transfer to at least one pollen-receiving plant.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of a pollination assembly according to an example embodiment of the present disclosure, with the pollination assembly shown coupled to a tractor;

FIG. 2 is another perspective view of the pollination assembly of FIG. 1, with part of the tractor removed and part of an air system of the pollination assembly removed;

FIG. 3 is a front elevation view of the pollination assembly of FIG. 2;

FIG. 4 is a top plan view of the pollination assembly of FIG. 2;

FIG. 5 is a side elevation view of the pollination assembly of FIG. 2;

FIG. 6 is a side elevation view of a duct unit of the pollination assembly of FIG. 2;

FIG. 7 is the perspective view of the pollination assembly of FIG. 2, with the duct unit removed and illustrating a collection unit and a blower unit of the assembly;

FIG. 8 is a perspective view of the collection unit of the pollination assembly of FIG. 7;

FIG. 9 is a perspective view of a plant handling unit of the collection unit of FIG. 8;

FIG. 10 is a perspective view of the blower unit of the pollination assembly of FIG. 7;

FIG. 11 is a perspective view of an end portion of the duct unit of the pollination assembly of FIG. 7;

FIG. 12 is a side elevation view of the end portion of the duct unit of FIG. 11;

FIG. 13 illustrates an example flow of pollen produced by the pollination assembly of FIG. 1; and

FIG. 14 is a perspective view of a pollination assembly according to another example embodiment of the present disclosure, with the pollination assembly shown coupled to a rail system.

Corresponding reference numerals indicate corresponding parts throughout the several views of the drawings.

### DETAILED DESCRIPTION

Example embodiments will now be described more fully with reference to the accompanying drawings. The description and specific examples included herein are intended for purposes of illustration only and are not intended to limit the scope of the present disclosure.

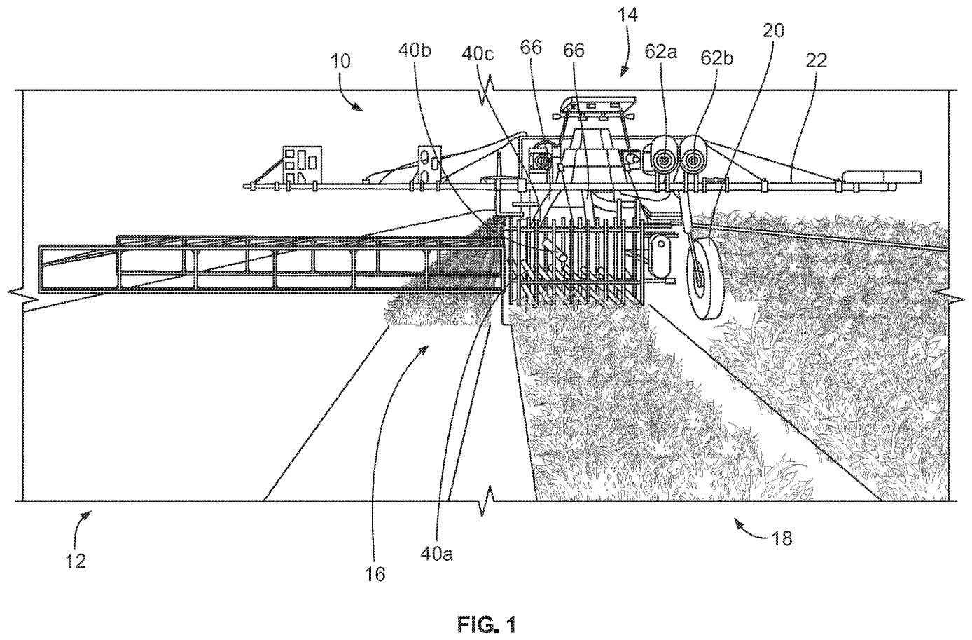

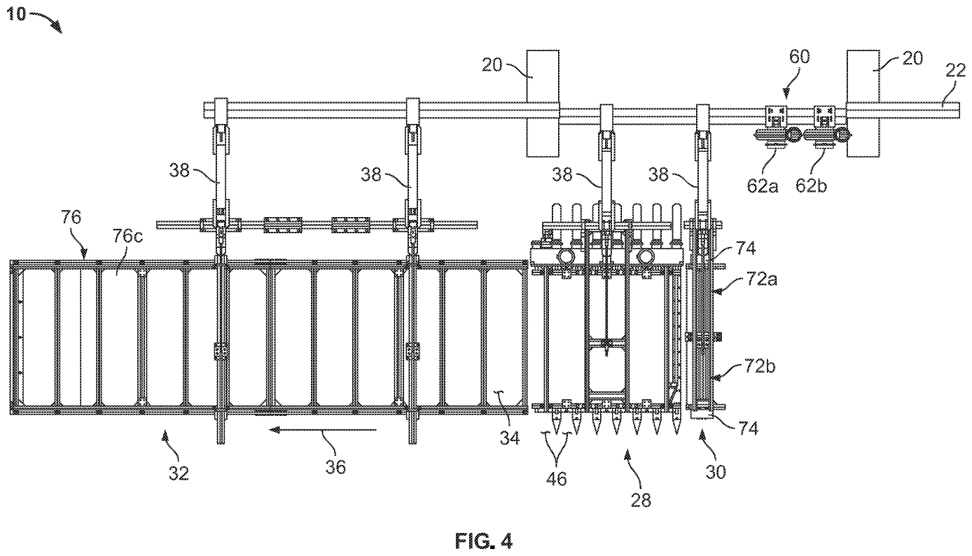

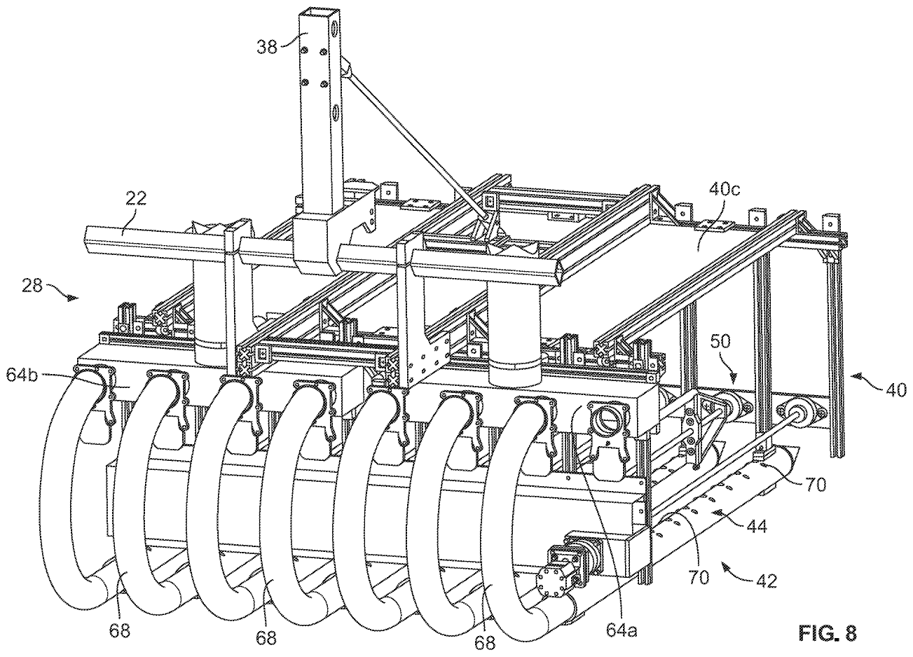

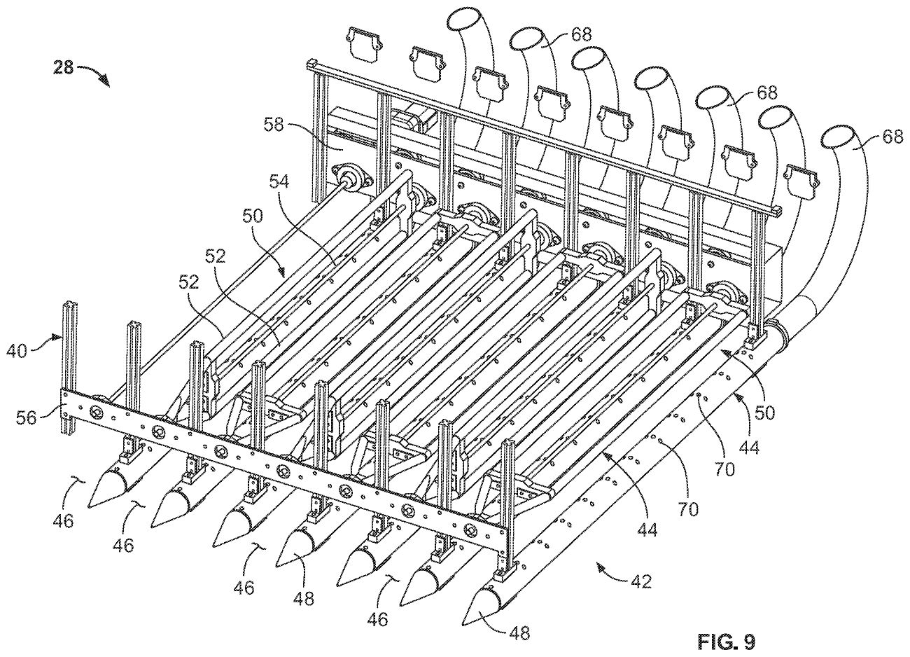

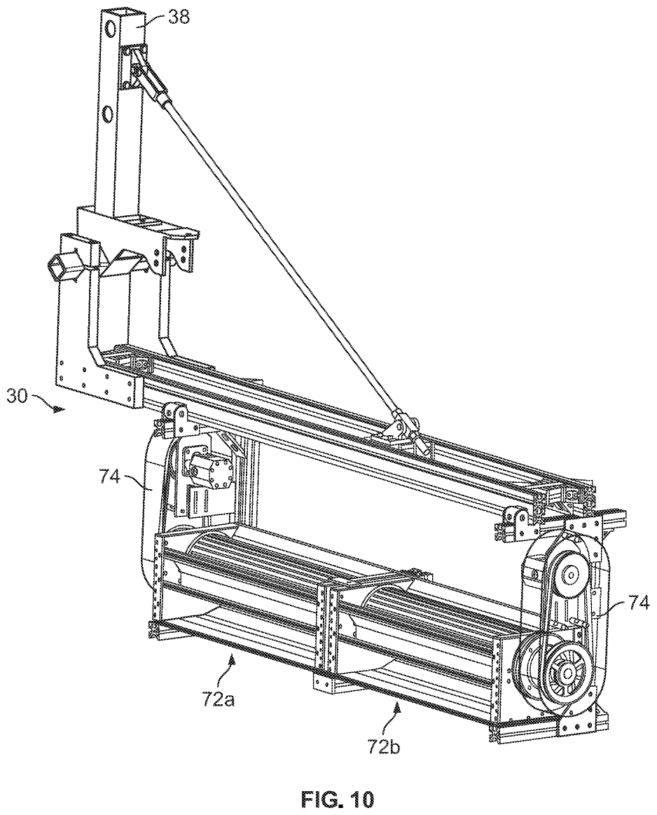

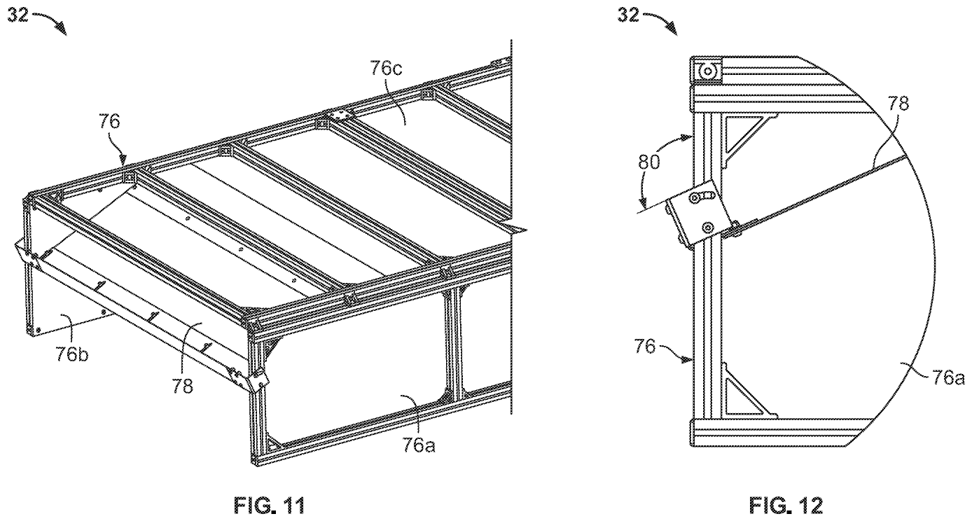

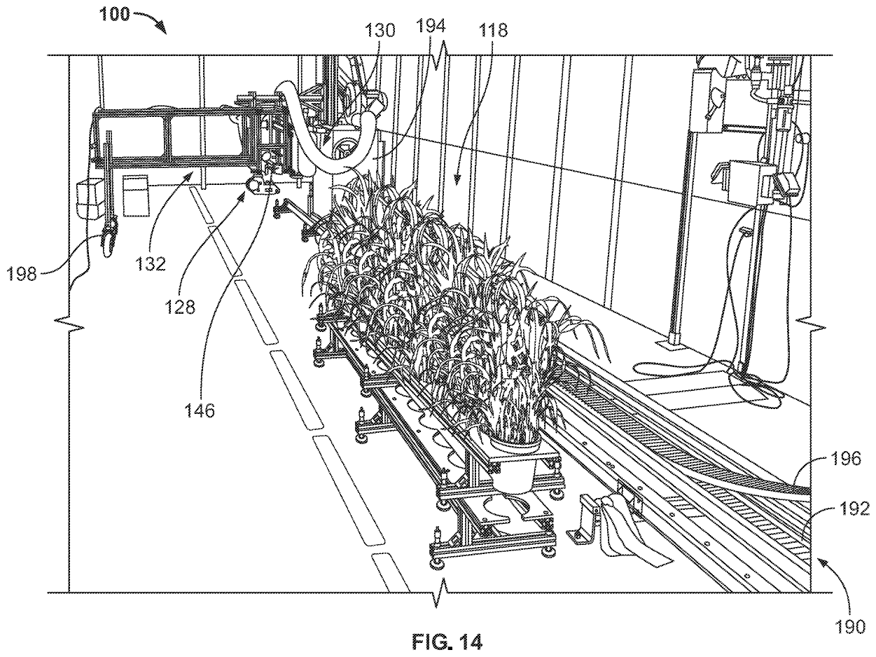

FIGS. 1-11 illustrate an example embodiment of a pollination assembly 10 including one or more aspects of the present disclosure. The pollination assembly 10 is configured to direct, move, deliver, convey, transfer, etc. pollen between plants, for example, for effecting pollination, etc. of the plants. In the illustrated embodiment, the pollination assembly 10 is shown in a field 12, in combination with (e.g., coupled to, mounted on, etc.) a tractor 14, whereby the pollination assembly 10 operates to transfer the pollen between plants in the field 12 as the tractor 14 moves through the field 12. The pollination assembly 10 and the tractor 14 may be viewed as a pollination system. In other embodiments, the pollination assembly 10 may be used independent of the tractor 14, for example, where the pollination assembly 10 may be moveable on a rail system, etc. to transfer pollen between plants in the field 12 (as the pollination assembly 10 moves through the field along the rails). In addition, in still other embodiments, the pollination assembly 10 may implemented/used in a greenhouse or other growing space to transfer pollen between plants therein (e.g., via a rail system, etc. in the greenhouse or other growing space (see, e.g., FIG. 14, etc.), etc.).

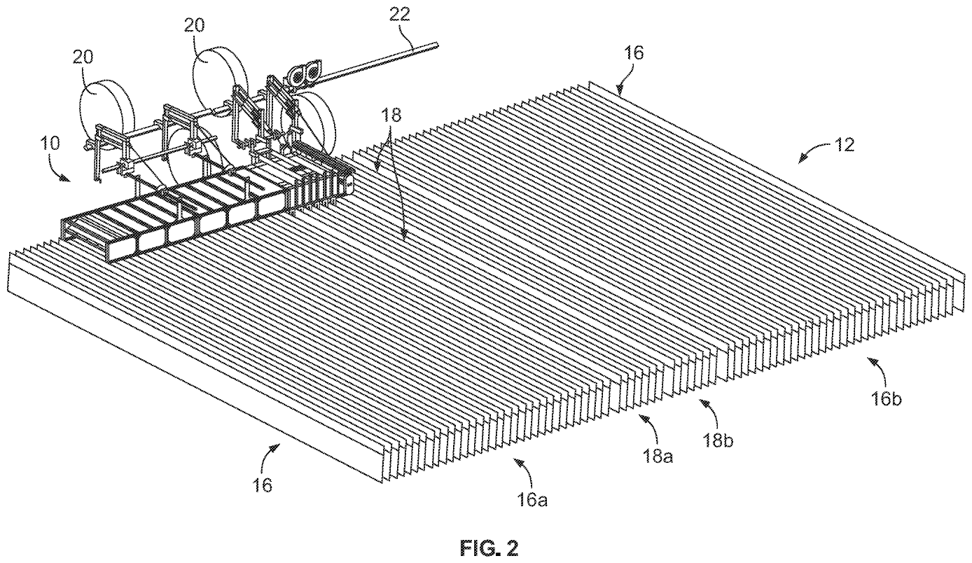

As shown in FIGS. 1 and 2, the field 12 includes multiple rows of pollen-receiving plants 16 and multiple rows of pollen-bearing plants 18. More particularly, the field 12 may include a group of rows of the pollen-bearing plants 18 (where the rows are grouped together) and a group of rows of the pollen-receiving plants 16 (where the rows are grouped together). For instance, the field 12 may include a group of seven rows of the pollen-bearing plants 18 (e.g., at a width of about 5 feet for the group, etc.) and then a group of thirty-two rows of the pollen-receiving plants 16 (adjacent the group of pollen-bearing plants 18) (e.g., at a width of about 20 feet for the group, etc.). This arrangement may be repeated as desired in the field 12, for example, so that a group of rows of pollen-bearing plants 18 is adjacent a group of rows of pollen-receiving plants 16. In the example illustrated in FIG. 2, the field 12 includes first and second groups 16a, 16b of pollen-receiving plants 16 and first and second groups 18a, 18b of pollen-bearing plants 18. In this arrangement, the group 18a of pollen-bearing plants 18 may be used to pollinate the group 16a of pollen-receiving plants 16, and the group 18b of pollen-bearing plants 18 may be used to pollinate the group 16b of pollen-receiving plants 16.

The rows of plants 16, 18 within each of the groups may be spaced as desired within the field 12 (e.g., the rows within the groups may be spaced apart by a distance of about 7.5 inches, by a distance of between about 6 inches and about 10 inches, by a distance of more than about 10 inches, by a distance of less than about 6 inches, etc.). In addition, the different groups of plants may be spaced apart as desired, for example, to accommodate (and receive) wheels 20 of the tractor 14 therebetween (e.g., an end row of a group of the pollen-bearing plants 18 may be spaced apart from an adjacent end row of a group of the pollen-receiving plants by a distance of about 1 foot or more or less, etc.). That said, it should be appreciated that the field 12 may include other groups with other numbers of rows of pollen-bearing plants 18 and/or pollen-receiving plants 16 and/or other spacings of the rows in other embodiments.

In this example embodiment, the plants 16, 18 include wheat plants. In addition, the pollen-receiving plants 16 have been emasculated or otherwise modified such that the pollen-receiving plants 16 cannot pollinate themselves or other plants. Such emasculation may be done mechanically, chemically, or genetically, etc. After the pollen-receiving plants 16 are emasculated, they still can be pollinated by the pollen of the pollen-bearing plants 18. It should be appreciated that the pollination assembly 10 may be used with other plants, other than wheat, within the scope of the present disclosure, for example, corn, canola, tomato, eggplant, sweet and hot peppers, amaranth, barley, oat, rye, wild rice, walnut, pecan, cabbage, broccoli, spinach, other suitable plants, etc. In addition, in some example embodiments, the pollen-bearing plants 18 may include a first variety of plant and the pollen-receiving plants 16 may include a second variety of the same plant. As such, when the pollen-receiving plants 16 are pollinated with pollen from the pollen-bearing plants 18, the pollen-receiving plants 16 produce cross-pollinated seeds that may be used for growing a cross-pollinated variety of the crop plant with certain altered characteristics.

That said, in this example, the tractor 14 is configured to carry the pollination assembly 10 through the field 12 to pollinate the pollen-receiving plants 16 (e.g., in group 16a, etc.) with the pollen collected from the pollen-bearing plants 18 in an adjacent group of rows (e.g., in group 18a, etc.). In particular, the tractor 14 is positioned in the field 12 to drive along, or in the same direction as, the rows of the plants 16, 18, while supporting the pollination assembly 10 (e.g., with the wheels 20 of the tractor 14 positioned in the larger spacing between the group of the pollen-receiving plants 16 and the group of the pollen-bearing plants 18, etc.). The pollination assembly 10, then, is configured to collect, receive, obtain, etc. pollen from the pollen-bearing plants 18 and direct (and deliver/transfer) the pollen to the adjacent pollen-receiving plants 16 (e.g., to female flowers of the pollen-receiving plants 16, etc.).

The illustrated tractor 14 has a front portion and a back portion and is configured to travel along the rows of the plants 16, 18 in a generally forward direction. The tractor 14 includes a carriage 22 (e.g., a tool bar, etc.) coupled to the tractor 14 adjacent the front portion. The pollination assembly 10, then, is configured to couple to (e.g., mount to, etc.) the tractor 14 at the carriage 22, whereby the pollination assembly 10 is configured to move along the rows of the plants 16, 18 with the tractor 14. The tractor 14 may travel through the field 12 at desired speeds, for example, between about 2 miles per hour (mph) and about 5 mph during operation. However, it should be appreciated that the tractor 14 may travel through the field 12 at other speeds within the scope of the present disclosure (e.g., at speeds greater than about 5 mph, etc.). In the illustrated embodiment, the tractor 14 includes a high clearance farm tractor such as an applicator from Hagie Manufacturing Company of Clairon, IA. However, other tractors, or other vehicles or machines more generally, suitable for carrying the pollination assembly 10 through the field 12 may be used in other embodiments. For example, in some embodiments, the pollination assembly 10 may be coupled to a carriage (broadly, a machine) arranged to move along a rail, etc.

While one pollination assembly 10 is shown coupled to the tractor 14 in FIGS. 1 and 2, it should be appreciated that an additional pollination assembly may also be coupled to the tractor 14, at the carriage 22 generally opposite the illustrated pollination assembly 10, in other embodiments. In this way, the tractor 14 may move through the field 12 and collect pollen from two adjacent groups of pollen-bearing plants 18 and direct (and deliver) the collected pollen to corresponding groups of pollen-receiving plants 16. For instance, with reference to FIG. 2, in such an example (where two pollination assemblies 10 are coupled to the tractor 14), a first pollination assembly may collect pollen from the first group 18a of pollen-bearing plants 18 and direct the pollen to the first group 16a of pollen-receiving plants 16, and a second pollination assembly may collect pollen from the second group 18b of pollen-bearing plants 18 and direct the pollen to the second group 16b of pollen-receiving plants 16.

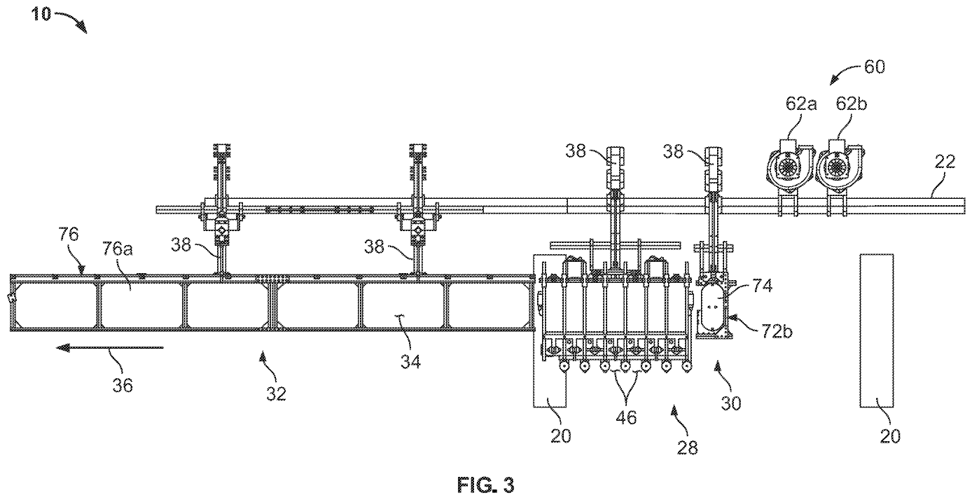

With additional reference to FIGS. 3-6, the pollination assembly 10 generally includes a collection unit 28, a blower unit 30, and a duct unit 32. As shown, the collection unit 28 is positioned generally between the blower unit 30 and the duct unit 32. In addition, the blower unit 30 is generally aligned (e.g., generally centrally from top to bottom, etc.) with a channel 34 extending generally through the collection unit 28 and the duct unit 32. In this arrangement, the collection unit 28 is configured to displace or release (or dislodge) pollen from the pollen-bearing plants 18 as the tractor 14 travels through the field 12. The blower unit 30 is configured to then direct air along the channel 34, generally through the collection unit 28 and into (and through) the duct unit 32, to direct the pollen into and through the duct unit 32. And, the duct unit 32 is configured to direct the pollen generally across the rows of the pollen-receiving plants 16 (in a direction indicated by arrow 36). In this way, the pollen released (or displaced) from the pollen-bearing plants 18 is directed, moved, delivered, conveyed, etc., by the duct unit 32, to the pollen-receiving plants 16 (e.g., to all or substantially all of the pollen-receiving plants 16 in the group adjacent the pollen-bearing plants 18, etc.) for pollination thereof.

The collection unit 28, the blower unit 30, and the duct unit 32 are each coupled to the carriage 22 of the tractor 14 by corresponding mounts 38 (e.g., lifts, booms, etc.). The mounts 38 are configured to support (and hold) the units 28, 30, 32 in desired positions relative to the tractor 14 and/or the plants 16, 18. In addition, the mounts 38 may be actuated, as desired (e.g., individually, as a group, etc.), to move the units 28, 30, 32 to desired positions relative to the tractor 14 and/or the plants 16, 18. For instance, the mounts 38 may be actuated to raise or lower one or more of the units 28, 30, 32 to properly position the units 28, 30, 32 relative to the plants 16, 18, depending on a height of the plants 16, 18, etc. Further, in some embodiments, such actuation may be automatic based on inputs received from one or more sensors coupled to the tractor 14 (configured for measuring, monitoring, etc. height of the plants 16, 18 as the tractor 14 moves through the field 12, etc.).

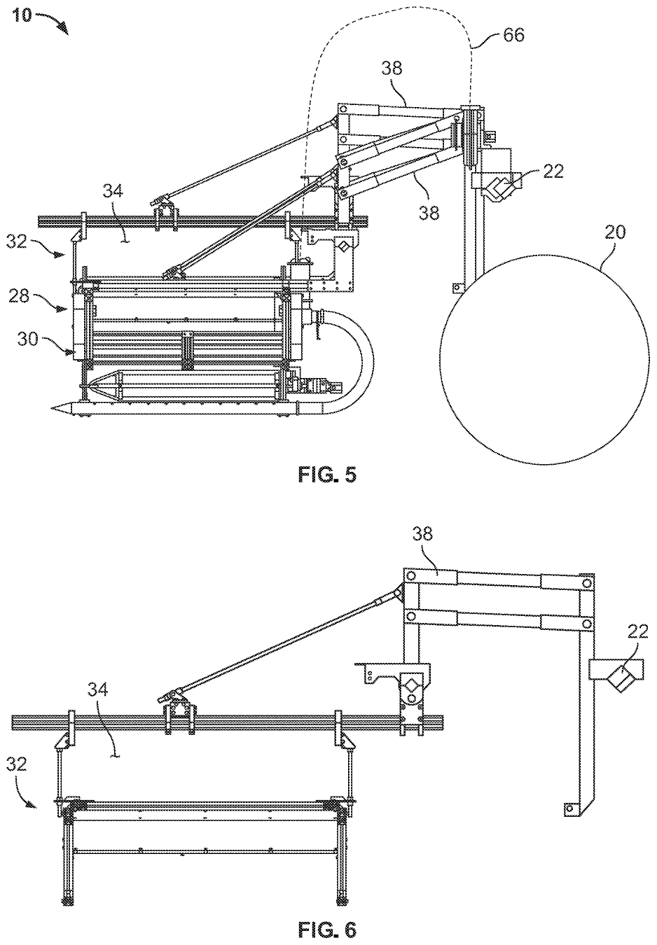

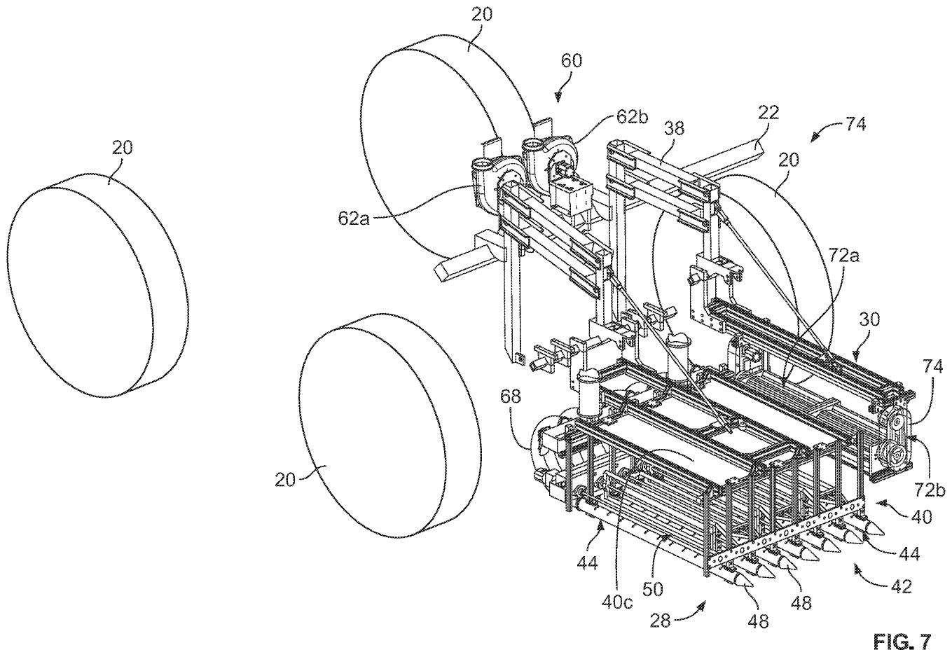

As further shown in FIGS. 7-9, the collection unit 28 generally includes a housing 40 and a plant handling unit 42 coupled to (and generally supported by) the housing 40. The housing 40 includes a forward wall 40a (FIG. 1), a rearward wall 40b (FIG. 1), and a top wall 40c (FIGS. 1 and 5). And, together, the walls 40a-c generally define the channel 34 extending through the housing 40. In one example (and without limitation), the housing 40 of the collection unit 28 may have a width of between about 1 foot and about 20 feet (e.g., about 2 feet, about 3 feet, about 3.75 feet, about 5 feet, about 6 feet, about 8 feet, about 10 feet, about 15 feet, etc.). That said, it should be appreciated that the housing 40 may have any desired and/or suitable length to accommodate sizes and/or positions of the pollen-receiving plants 16 and/or the pollen-bearing plants 18 in the field 12, etc. In addition, in various example embodiments, the walls 40a-c of the collection unit 28 may be constructed from suitable material such as, for example (and without limitation), metals, plastics, plexiglass, polycarbonates, ctc.

The plant handling unit 42 of the collection unit 28 includes multiple guides 44 arranged in a generally parallel configuration below the housing 40. The guides 44 extend generally in the forward direction of travel of the tractor 14 (during use of the pollination assembly 10). And, passages 46 are defined by the guides 44 generally within the housing 40 (e.g., adjacent the guides 44, between adjacent ones of the guides 44, etc.). The guides 44 are configured (e.g., oriented, sized, shaped, spaced, etc.) to receive and/or direct the pollen-bearing plants 18 into the passages 46, and then into the housing 40, as the tractor 14 moves the collection unit 28 through the field 12. Additionally, a forward end portion 48 (or tip, etc.) of each of the guides 44 is generally conical in shape to help direct the plants 18 into the corresponding passage 46. The guides 44, and the passages 46, are spaced (e.g., in a direction generally perpendicular to longitudinal axes of the guides 44, etc.) to generally match a spacing of (or between) the rows of the pollen-bearing plants 18 in the field 12. As such, as the tractor 14 drives forward through the field 12, each of the passages 46 generally aligns with one of the rows of the pollen-bearing plants 18 (such that plants 18 in each of the different rows may be received generally simultaneously into the housing 40 via the multiple passages 46). In the illustrated embodiment, the plant handling unit 42 includes seven guides 44 defining seven passages 46 (thereby generally corresponding to the seven rows of pollen-bearing plants 18 in each of the groups 18a and 18b in FIG. 2). In other embodiments, though, the plant handling unit 42 may include more than seven guides or fewer than seven guides defining a different number of passages within the scope of the present disclosure.

The plant handling unit 42 also includes multiple agitators 50 each associated with one of the passages 46. The agitators 50 are positioned generally above the guides 44 within the housing 40, and each is supported by the housing in general alignment with (e.g., above, etc.) one of the passages 46. In this arrangement, as the pollen-bearing plants 18 are received into the passages 46 of the plant handling unit 42, and move into the housing 40, the agitators 50 engage (e.g., operatively contact, etc.) the plants 18 to facilitate release of pollen therefrom. In the illustrated embodiment, the agitators 50 engage top portions of the pollen-bearing plants 18 (e.g., wheat heads (or head portions) of the plants 18 when the plants are wheat plants, etc.) to facilitate release of the pollen. That said, in general, each of the agitators 50 is positioned to contact and/or disturb the male flowers of the pollen-bearing plants 18 to release the pollen from the plants. It should be appreciated that the positions of the agitators 50 within the housing may be adjusted (e.g., automatically, etc.) to account for variations in the height of the heads and/or male flowers of the pollen-bearing plants 18, and/or to account for particular locations of the heads and/or mail flowers of the pollen-bearing plants 18 (e.g., depending on a type of the pollen-bearing plants 18, etc.), etc.

In the illustrated embodiment, each of the agitators 50 includes a pair of arms 52 coupled to a shaft 54 (e.g., a spindle, etc.) supported on the housing 40 via a forward bearing support 56 and a rearward drive 58 (e.g., a cogged belt drive/system, etc.). The arms 52, then, are configured to rotate via the shaft 54, through operation of the drive 58 to turn/rotate the shaft 54. In this manner, as the pollen-bearing plants 18 are received into the passages 46 and move into the housing 40, the arms 52 of the agitators 50 rotate and engage upper portions of the pollen-bearing plants 18 (within the housing 40). In this example, the arms 52 of each of the agitators are arranged to define a generally rectangular shape. In addition, the arms 52 are spaced apart from each other by a desired distance (e.g., a distance between about 3 inches and about 9 inches, a distance of about 6 inches, etc.) to thereby provide an agitation zone in which the arms contact the pollen-bearing plants 18. Further, the drive 58 of the plant handling unit 42 is configured to rotate the arms 52 of each of the agitators 50 at desired speeds (e.g., so long as to inhibit damage to plants 16, 18, etc.), for example, between about 100 rotations per minute (rpm) and about 500 rpm, at a speed of about 200 rpm, at a speed of about 300 rpm, at a speed of less than about 100 rpm, at a speed of greater than about 500 rpm, etc. As such, the arms 52 are configured to engage each of the plants 18 (e.g., within the agitation zone of each of the agitators 50, etc.) multiple times while the plants 18 are present in the housing 40 to dislodge, etc. pollen therefrom. It should be appreciated that the agitators 50 may include other configurations in other embodiments, for example, other arrangements of arms than illustrated (e.g., a single arm, three arms, four arms, more than four arms, arms arranged in other than rectangular configurations, etc.), configurations that do not include arms (e.g., chains, etc.), etc.

With continued reference to FIGS. 7-9, the pollination assembly 10 includes an air system 60 coupled to the plant handling unit 42 for use in moving pollen removed from the pollen-bearing plants 18 (by the agitators 50) into the housing (and/or for temporarily maintaining the removed pollen in the housing, etc.). The air system 60 generally includes blowers 62a, 62b mounted on the carriage 22 of the tractor 14, and manifolds 64a, 64b mounted on the housing 40 of the plant handling unit 42. The blowers 62a, 62b are each coupled to a corresponding one of the manifolds 64a, 64b via conduits 66 (see, also, FIGS. 1 and 5). Additionally, the guides 44 of the collection unit 28 are each formed by a tube coupled to one of the manifolds 64a, 64b via a corresponding hose 68. And, multiple nozzles 70, then, are defined in each of the guides 44 (e.g., in two rows along a top portion of the guide 44, etc.). In connection therewith, the blowers 62a, 62b are each configured to generate and provide a flow of air to (or at) the guides 44, such that a speed of air discharged from the nozzles 70 may be a desired or suitable speed (e.g., between about 2 meters per second (mps) and about 30 mps (e.g., about 3 mps, about 4 mps, about 5 mps, about 6 mps, about 6.5 mps, about 7 mps, about 8 mps, about 9 mps, about 10 mps, about 12 mps, about 14 mps, about 20 mps, about 25 mps, etc.), etc.).

In the illustrated embodiment (and without limitation), three of the guides 44 are coupled to the manifold 64a (e.g., a left three of the guides as viewed in FIG. 7, etc.), and four of the guides 44 are coupled to the manifold 64b (e.g., a right four of the guides as viewed in FIG. 7, etc.). The blowers 62a, 62b, then, are configured to generate and direct air to the corresponding manifolds 64a, 64b (via the conduits 66), and the manifolds 64a, 64b distribute the air to the corresponding ones of the guides 44 coupled thereto. Valves are provided within the manifolds 64a, 64b, in connection with each of the guides 44, to control, balance, etc. air flow received from the blowers 62a, 62b into the individual ones of the guides 44 (e.g., to produce, provide, etc. a desired speed of air discharge from the nozzles 70 at each of the guides 44, etc.). In turn, the nozzles 60 are configured to direct the air received in the guides 44 in a generally upward direction within the housing 40. As such, as the pollen is dislodged from the pollen-bearing plants 18 by the agitators 50, the air from the nozzles 70 (located generally below the agitators 50) helps lift (and/or direct) the pollen out of a canopy of the plants 18 and generally upward into the housing 40 (e.g., into an upper portion of the housing 40, etc.) whereby the dislodged pollen may subsequently be directed to the pollen-receiving plants 16 without interference from the pollen-bearing plants 18 (e.g., without inadvertently being blown back into the pollen-bearing plants 18, etc.).

With reference now to FIGS. 3-4, 7, and 10, the blower unit 30 of the pollination assembly 10 is positioned adjacent the housing 40 of the collection unit 28 and includes blowers 72a, 72b (e.g., fan blowers, etc.) configured to produce a flow of air (via motors 74) and to then direct the flow of air into the housing 40 (e.g., generate a lateral airflow through the housing 40 relative to the upward airflow generated by the guides 44, etc.). The blowers 72a, 72b are each generally aligned with an upper portion of the housing 40 (e.g., a portion of the housing 40 generally above the agitators 50, etc.). As such, the blowers 72a, 72b operate to direct (via the generated flow of air) the dislodged pollen from the housing 40 (as pushed upward in the housing by the guides 44) toward and into (and through) the duct unit 32 (e.g., along and/or through the channel 34 generally defined by the housing 40 and the duct unit 32, etc.). In this way, the air system 60 (via the nozzles 70) and the blower unit 30 operate to create an airstream or airflow along the channel 34 in the pollination assembly 10 (e.g., via the Coandă effect, etc.) to direct and deliver the pollen dislodged from the pollen-bearing plants 18 to the pollen-receiving plants 16 (which are located generally under and/or generally in alignment with the duct unit 32 for such delivery).

In example embodiments, the blowers 72a, 72b may be configured to generate an air flow having an air speed of between about 10 mps and about 30 mps (e.g., about 12 mps, about 14 mps, about 16 mps, about 18 mps, about 20 mps, about 25 mps, etc.), for instance, to generate and/or produce a desired blowing distance of the dislodged pollen (e.g., between about 5 feet and about 50 feet, about 10 feet, about 15 feet, about 20 feet, about 30 feet, etc.). In connection therewith, the desired air flow of the blowers 72a, 72b and/or desired blowing distance of the pollen may be based on a length of the duct unit 32, a number of rows of the pollen-receiving plants 16 to receive the pollen, the type of the plants 16, 18 and/or pollen collected therefrom, etc. In addition, in some example embodiments, the blowers 72a, 72b may be adjusted (e.g., tilted or angled, etc.) to direct the flow of air from the blowers 72a, 72b generally toward the upper portion of the housing 40 (e.g., at an upward angle of about 1 degree, about 2 degrees, about 3 degrees, about 4 degrees, about 5 degrees, etc.).

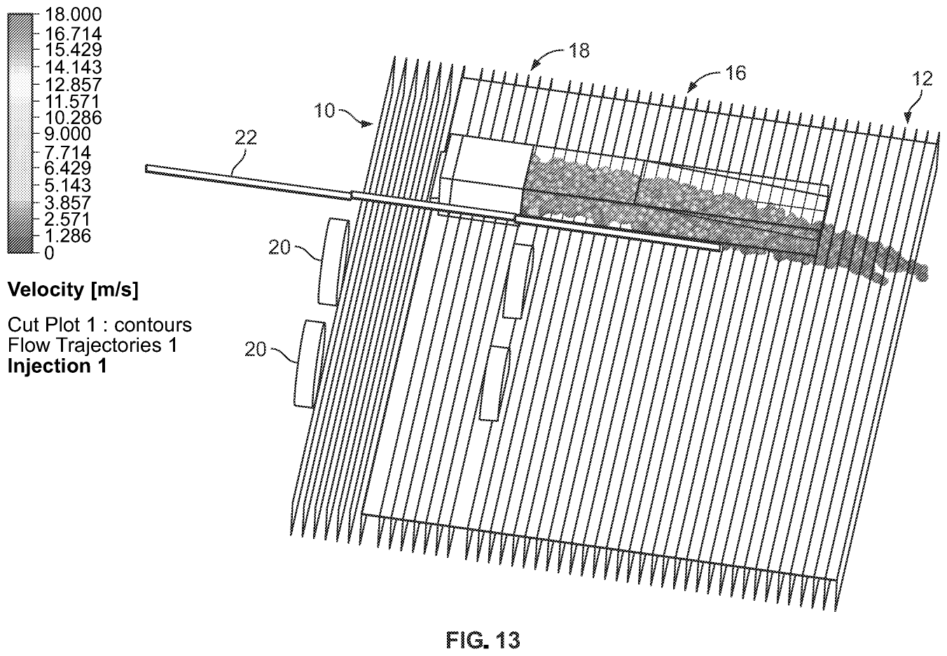

Referring now to FIGS. 3-4 and 11-12, the duct unit 32 includes an elongate body 76 extending generally laterally away from the collection unit 28. The body 76 includes a front wall 76a, a rear wall 76b, and a top wall 76c. In this arrangement, the walls 76a-c define the portion of the channel 34 extending generally away from the collection unit 28, and through the duct unit 32, for directing, carrying, etc. the dislodged pollen to and generally over the pollen-receiving plants 16 in the field 12. A bottom side/portion of the body 76 (and channel 34) is open (e.g., does not include a wall, etc.) to facilitate movement of the pollen to the pollen-receiving plants 16 as the duct unit 32 passes thereover (and as the pollen loses speed and begins to drop/fall out of the air flow through the duct unit 32). An end portion of the body 76 is also open, to allow pollen to exit the channel 34 and deliver pollen to pollen-receiving plants 16 adjacent to the duct unit 32. In one example (and without limitation), the body 76 of the duct unit 32 may have a length of between about 5 feet and about 30 feet (e.g., about 10 feet, about 15 feet, about 16 feet, about 20 feet, about 25 feet, etc.). In addition, in various example embodiments, the walls 76a-c of the body 76 may be constructed from suitable material such as, for example (and without limitation), metals, plastics, plexiglass, polycarbonates, etc.

In the illustrated embodiment, the body 76 of the duct unit 32 includes a baffle 78 disposed at the end portion of the top wall 76c (e.g., defined by the top wall 76c, coupled to the top wall 76c, etc.). The baffle 78 is oriented at a generally downward angle 80 to direct the pollen generally down and toward the pollen-receiving plants 16 as the pollen exits the duct unit 32. For instance, the angle 80 formed by baffle 78 may be between about 90 degrees and about 135 degrees (e.g., about 100 degrees, about 110 degrees, about 114 degrees, about 120 degrees, about 125 degrees, etc.).

In an example operation, the tractor 14 and pollination assembly 10 may be directed to the field 12 having the pollen-receiving plants 16 and pollen-bearing plants 18 planted (in the groups 16a, 16b, 18a, 18b) as described above (and as illustrated in FIG. 2) (e.g., when a pollination window starts for the plants 16, 18, etc.). A position of the pollination assembly 10 on the tractor 14 may then be adjusted/set based on height(s) of the pollen-bearing plants 18 and the pollen-receiving plants 16 (e.g., manually, automatically using height sensors on the tractor 14 and/or pollination assembly 10, etc.). The tractor 14 is then driven through the field 12, with the passages 46 of the plant handling unit 42 generally aligned with the rows of the pollen-bearing plants 18.

As the tractor 14 moves, the pollen-bearing plants 18 enter the housing 40 of the collection unit 28. In turn, the agitators 50 engage upper portions of the plants 18 and dislodge pollen therefrom. At the same time, the nozzles 70 of the guides 44 direct the dislodged pollen upward in the housing 40 and out of the canopy of the plants 18 (via air flow from the air system 60). And, the blowers 62a, 62b then direct the pollen into body 76 of the duct unit 32. And, as the pollen moves through the duct unit 32, some begins to fall (or settle) onto the pollen-receiving plants 16 located thereunder (for pollination) and the rest generally exits the duct unit 32 adjacent the baffle 78 for delivery to the pollen-receiving plants 16 adjacent the end portion of the duct unit 32. As such, in this example, pollen from the seven rows of pollen-bearing plants 18 is directed, via the pollination assembly 10, to all or substantially all of the 32 rows of pollen-receiving plants 16 planted adjacent the pollen-bearing plants 18.

In connection with the above operation, FIG. 13 illustrates an example flow of the pollen through the pollination assembly 10, from the pollen-bearing plants 18 to the adjacent pollen-receiving plants 16. As shown, the unique combination of features included in the pollination assembly 10 (e.g., the collection unit 28, the blower unit 30, duct unit 32, the air system 60, etc.) facilitate a generally uniform distribution of the pollen dislodged from the pollen-bearing plants 18 generally laterally across the adjacent rows of pollen-receiving plants 16 (e.g., without vertical loss/dissipation of the pollen, without rearward loss/dissipation of the pollen, without loss of the pollen in the canopy of the pollen-bearing plants 18, etc.).

FIG. 14 illustrates a pollination assembly 100 according to another example embodiment of the present disclosure. The pollination assembly 100 of this embodiment is substantially similar to the pollination assembly 10 described above with reference to FIGS. 1-12. For example, the pollination assembly 100 generally includes a collection unit 128, a blower unit 130, and a duct unit 132 each of which is substantially the same as described above for the pollination assembly 10. In addition, the pollination assembly 100 is configured to direct, move, deliver, convey, etc. pollen between plants, for example, for effecting pollination, etc. of the plants in substantially the same way as described above for the pollination assembly 10.

That said, the pollination assembly 100 of this embodiment is configured to move relative to pollen-receiving plants (not shown) and pollen-bearing plants 118 along a rail system 190. In connection therewith, the rail system 190 includes a rail 192 configured to guide the pollination assembly 100 (via a carriage 194 (or carrier) coupled to the pollination assembly 100 and disposed on the rail 192) in a desired direction relative to the plants. And, a belt drive 196 is provided to drive the pollination assembly 100 (via the carriage 194) along the rail 192 (e.g., such that the pollen-bearing plants 118 can be received in a passage 146 of the collection unit 128 to facilitate removal of pollen therefrom (in the same manner described above for the assembly 10), etc.). Wheels 198 are provide adjacent an end of the duct unit 132 to provide support thereto and help facilitate the movement of the pollination assembly 100. In this example embodiment, the pollination assembly 100 and the rail system 190 may be viewed as a pollination system.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

Example embodiments have been provided so that this disclosure will be thorough, and will fully convey the scope to those who are skilled in the art. Numerous specific details are set forth such as examples of specific components, assemblies, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Specific dimensions, specific materials, and/or specific shapes disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, components, and/or groups thereof, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” and the phrase “at least one of” includes any and all combinations of one or more of the associated listed items.

Although the terms first, second, third, etc. may be used herein to describe various elements, components, seeds, members and/or sections, these elements, components, seeds, members and/or sections should not be limited by these terms. These terms may be only used to distinguish one element, component, seed, member or section from another element, component, seed, member or section. Terms such as “first,” “second,” and other numerical terms when used herein do not imply a sequence or order unless clearly indicated by the context. Thus, a first element, component, seed, member or section discussed below could be termed a second element, component, seed, member or section without departing from the teachings of the example embodiments.

Spatially relative terms, such as “inner,” “outer,” “beneath,” “below,” “lower,” “above,” “upper,” and the like, may be used herein for case of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. A pollination assembly for use in collecting and transferring pollen between plants, the pollination assembly comprising:
a collection unit configured to dislodge pollen from pollen-bearing plants;
a duct unit disposed adjacent the collection unit, wherein the collection unit and the duct unit define a channel extending through the collection unit and the duct unit; and
a blower unit configured to direct air into the collection unit and the duct unit to thereby transport the pollen dislodged from the pollen-bearing plants along the channel to pollen-receiving plants adjacent the duct unit.

2. The pollination assembly of claim 1, wherein the collection unit defines at least one passage for receiving the pollen-bearing plants into the collection unit.

3. The pollination assembly of claim 2, further comprising at least one agitator configured to engage the pollen-bearing plants received into the collection unit to thereby dislodge the pollen from the pollen-bearing plants.

4. The pollination assembly of claim 3, wherein the at least one agitator includes at least two arms; and/or
wherein the at least one passage includes multiple passages each configured to receive the pollen-bearing plants into the collection unit.

5. (canceled)

6. The pollination assembly of claim 1, wherein the blower unit includes at least one fan configured to generate the air and direct the air into the collection unit and the duct unit.

7. The pollination assembly of claim 1, wherein the duct unit includes a body having a front wall, a rear wall, a top wall, and an open bottom.

8. The pollination assembly of claim 7, wherein the body of the duct unit includes a first end portion disposed adjacent the collection unit and a second, opposite end portion, wherein the duct unit includes a baffle disposed within the body, and wherein the baffle is angled generally downward relative to the top wall of the body.

9. The pollination assembly of claim 1, wherein the collection unit includes at least one guide configured to facilitate movement of the pollen-bearing plants into the collection unit.

10. The pollination assembly of claim 9, further comprising an air system in fluidic communication with the at least one guide of the collection unit to provide air to the at least one guide;
wherein the at least one guide is further configured to direct the air from the air system into the collection unit for generally positioning the pollen in the channel within the collection unit.

11. (canceled)

12. The pollination assembly of claim 1, wherein the collection unit includes at least one guide configured to facilitate movement of the pollen-bearing plants into the collection unit; and
wherein the pollination assembly further includes at least one agitator disposed generally above the at least one guide, the at least one agitator configured to engage the pollen-bearing plants received into the collection unit to thereby dislodge the pollen from the pollen-bearing plants.

13. The pollination assembly of claim 12, further comprising an air system in fluidic communication with the at least one guide of the collection unit to provide air to the at least one guide;
wherein the at least one guide is further configured to direct the air from the air system toward the at least one agitator for generally positioning the pollen dislodged from the pollen-bearing plants in the channel within the collection unit.

14. A tractor comprising the pollination assembly of claim 1.

15. A pollination system, comprising:
a tractor having a carriage; and
the pollination assembly of claim 1 coupled to the carriage of the tractor.

16. A pollination system, comprising:
a rail system having at least one rail and a carriage configured for movement along the at least one rail relative to the at least one pollen-bearing plant; and
the pollination assembly of claim 1 coupled to the carriage and moveable, via the carriage, along the at least one rail.

17. The pollination system of claim 16, further comprising a greenhouse including the at least one pollen-bearing plant;
wherein the at least one rail is disposed in the greenhouse.

18. A method for collecting and transferring pollen between plants, the method comprising:
receiving at least one pollen-bearing plant into a housing of a collection unit of a pollination assembly;
dislodging, by at least one agitator, pollen from the at least one pollen-bearing plant within the housing;
directing, by a first air stream, the dislodged pollen from the pollen-bearing plant in an upward direction within the housing; and
directing, by a second air stream, the dislodged pollen from the housing into a duct unit adjacent the housing of the collection unit for transfer to at least one pollen-receiving plant.

19. The method of claim 18, further comprising positioning the duct unit over the at least one pollen-receiving plant and delivering the dislodged pollen to the at least one pollen-receiving plant while the duct unit is positioned over the at least one pollen-receiving plant.

20. The method of claim 18, wherein receiving the at least one pollen-bearing plant into the housing of the collection unit includes directing, by at least one guide, the at least one pollen-bearing plant into a passage of the collection unit and then receiving the at least one pollen-bearing plant into the housing through the passage.

21. The method of claim 20, further comprising generating the first air stream at the at least one guide.

22. The method of claim 18, wherein dislodging, by the at least one agitator, the pollen from the at least one pollen-bearing plant within the housing includes contacting the at least one pollen-bearing plant with the at least one agitator.

23.-29. (canceled)
